(19) United States
(12) Patent Application Publication
(10) Pub. No.: US 2019/0143055 A1
(43) Pub. Date: May 16, 2019

(54) APPARATUS FOR AND METHODS OF ADMINISTERING SUBSTANCES INTO AN INHALATION FLOW PATH

(71) Applicant: CLERISY CORPORATION, Rochester, NY (US)

(72) Inventors: Mary MAIDA, Canandaigua, NY (US); Patsy B REED, Sedona, AZ (US)

(21) Appl. No.: 16/098,887

(22) PCT Filed: May 5, 2016
(86) PCT No.: PCT/US2016/030887

§ 371 Date: Nov. 5, 2018

(51) Int. Cl. A61M 15/08

(52) U.S. Cl. CPC A61M 15/085; A41D 13/11

(57) ABSTRACT

A method of introducing a vapor of at least one substance into an inhalation flow path of a respiratory organ is provided. The method describes providing a carrier conformable to a surface of skin; providing the carrier with at least one substance; providing a barrier coupled to a surface of the carrier, the barrier being substantially impermeable to the substances carried by the carrier; providing a polyethylene coated material disposed within or atop the barrier and configured as a clamp for a septum of a user, wherein the polyethylene coated material is heat sealed within the barrier; and engaging the carrier to the surface of skin proximate an inhalation flow path of a respiratory organ with the barrier adapted to be interposed between the carrier and the skin to prevent the one or more substances from contacting the skin; wherein the substance is non-volatile. A vehicle and mask for introducing a vapor are also provided.

### CROSS REFERENCE TO RELATED APPLICATION

This application claims the benefit of PCT application No. PCT/US16/30887, filed May 5, 2016, the entire contents of which are incorporated by reference herein.

### INCORPORATION BY REFERENCE

The following patent is incorporated herein by reference as background information for this application: U.S. Pat. No. 6,295,982: Fred Dewitt Reed, Jr. Original Assignee Fred Dewitt Reed, Jr.; Publication date 2001-10-02; Grant date 2001-10-02.

### FIELD OF THE INVENTION

This invention relates generally to the field of aromatherapy and, more particularly, to vehicles for and methods of introducing one or more substances, both volatile and/or non-volatile, into an inhalation flow path of a user.

### BACKGROUND OF THE INVENTION

People have been introducing substances into inhalation flow path for inhalation into the nasal, olfactory, lungs or respiratory tract for therapeutic needs, for hundreds, if not thousands of years. Aromatherapy, as an example, utilizes introduction of volatile substances such as essential oils provided from, for instance, flowers, herbs and other selected plant life, fruits, and the like. Because the vapor of these and other potential varieties of volatile essential oils prove to provide users with beneficial physiological and psychological benefits, aromatherapy has become increasingly popular throughout the world.

To this end, items such as pillows, earrings and other forms of jewelry, diffusers, steam vaporizers, candles, masks, soaps, ointments and salves have been devised as vehicles for containing volatile substances and for providing users with the vapor from the volatile substances.

Other means for introduction of volatile substances have also been described, as in for example U.S. Pat. No. 6,295,982, incorporated herein by reference. This patent describes a vehicle for introducing a vapor of one at least one volatile substance into an inhalation flow path of a respiratory organ, comprising a carrier conformable to a surface of skin, at least one volatile substance carried by the carrier, a barrier coupled to a surface of the carrier, the barrier being substantially impermeable to the at least one volatile substances carried by the carrier; and means for engaging the carrier is a surface of skin proximate an inhalation flow path of the respiratory organ with the barrier adapted to be interposed between the carrier and the surface of skin to prevent the at least one volatile substances from contacting the skin.

However, the aforementioned approaches are generally not suitable for delivering non-volatile substances, and may, in some cases be unduly uncomfortable to the user. Furthermore, past approaches are unduly burdensome in that they require additional or supplementary steps to deliver the substance to the user.

Accordingly, a method and system for delivering a substance to user is desired.

### SUMMARY OF THE INVENTION

To achieve the forgoing and other aspects and in accordance with the purpose of the invention, apparatus for and methods of administering substances, both volatile and/or non-volatile, into an inhalation flow path is presented.

Accordingly, it is an object of the present invention to provide new and improved vehicles for and methods of introducing a vapor of one or more substances, both volatile and/or non-volatile, into an inhalation flow path.

Another object of the present invention is to provide new and improved vehicles for introducing a vapor of one or more substances, both volatile and/or non-volatile, into an inhalation flow path that are easy to construct.

Another object of the present invention is to provide new and improved vehicles for introducing a vapor of one or more substances, both volatile and/or non-volatile, into an inhalation flow path that are inexpensive.

Still another object of the present invention is to provide new and improved vehicles for introducing a vapor of one or more substances, both volatile and/or non-volatile, into an inhalation flow path that are unobtrusive and comfortable during use.

Yet another object of the present invention is the provision of providing users with convenient vehicles for introducing a vapor of one or more substances, both volatile and/or non-volatile, into an inhalation flow path that may be easily transported and used at any desired moment.

A further object of the present invention is to provide new and improved vehicles for and methods of introducing a vapor of one or more volatile and/or non-volatile substances into an inhalation flow path that are highly efficient and mitigate the number of steps a user must take in order to be supplied the substance.

As such, a method of introducing a vapor of at least one substance into an inhalation flow path of a respiratory organ is disclosed. The method comprises the steps of providing a carrier conformable to a surface of skin; providing the carrier with at least one substance; providing a barrier coupled to a surface of the carrier, the barrier being substantially impermeable to the substances carried by the carrier; providing a coated material disposed within or atop the barrier and configured as a clamp for a septum of a user, wherein the polyethylene coated material is heat sealed within the barrier; and engaging the carrier to the surface of skin proximate an inhalation flow path of a respiratory organ with the barrier adapted to be interposed between the carrier and the skin to prevent the one or more substances from contacting the skin; wherein the substance is non-volatile.

A vehicle for introducing a vapor of one at least substance into an inhalation flow path of a respiratory organ is also disclosed. The vehicle comprises a carrier conformable to a surface of skin; at least one substance carried by the carrier; a barrier coupled to a surface of the carrier, the barrier being substantially impermeable to the at least substance carried by the carrier; and a coated material disposed within or atop the barrier and configured as a clamp for a septum of a user, wherein the polyethylene coated material is heat sealed within the barrier; wherein the substance is non-volatile.

A mask for introducing a vapor of one at least substance into an inhalation flow path of a respiratory organ is also discussed. The mask comprises a first layer comprising a permeable layer; a second layer configured to carry at least one substance, and also act as a permeable barrier; a third layer comprising a substantially impermeable barrier to the at least substance carried by the carrier; and wherein the substance is activatable.

Other features, advantages, and aspects of the present invention will become more apparent and be more readily understood from the following detailed description, which should be read in conjunction with the accompanying drawings.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is an exploded perspective view of a vehicle for introducing a vapor of one or more volatile and/or non-volatile into an inhalation flow path in accordance with the present invention;

FIG. 2 is a perspective view of an embodiment of a vehicle for introducing a vapor of one or more volatile and/or non-volatile substances into an inhalation flow path in accordance with the present invention;

FIG. 3 is a side view of the the vehicle of FIG. 1 and FIG. 2;

FIG. 4 is a perspective view of an embodiment of a vehicle for introducing a vapor of one or more volatile and/or non-volatile substances into an inhalation flow path, the vehicle shown as it would appear worn by a user, in accordance with the present invention;

FIG. 5 a perspective view of another embodiment of a vehicle for introducing a vapor of one or more volatile and/or non-volatile substances into an inhalation flow path, the vehicle shown as it would appear worn by a user, in accordance with the present invention;

FIG. 6 is a side elevational view of the vehicle of FIG. 1 in accordance with the present invention;

FIG. 7 is a side elevational view of a vehicle in accordance with embodiments the present invention;

FIG. 8 is a side elevational view of a vehicle in accordance with embodiments of the present invention;

FIG. 9 is a top elevational view of the assembly of FIG. 8;

FIG. 10 is an exploded perspective view of a vehicle in accordance with an embodiment of the present invention; and

FIG. 11 is a perspective view of a vehicle for introducing a vapor of a substance in accordance with an embodiment of the present invention.

Unless otherwise indicated illustrations in the figures are not necessarily drawn to scale.

### DETAILED DESCRIPTION OF THE PREFERRED EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

As used herein, the term “microspheres” are defined as small capsules used to contain volatile and/or non-volatile substances and render them temporarily inactive. In embodiments of the present invention, they are adapted to diffuse the volatile and/or non-volatile substance into the surrounding air passing to the benefit of the user, and are of a diameter lying in the range 0.5 μm to 20 μm. By means of these dispositions, it is possible to diffuse volatile and/or non-volatile substances described herein.

As used herein, the term “non-volatile substance” refer to substances that do not readily evaporate into a gas under existing conditions, but rather evaporate upon a triggering event and may be in the form of powders or in gel-based microsphere. They may be referred to as “relatively non-volatile”, and may comprise Vitamin D Non-volatile plant substances are mainly derived from the plan seeds or can be chemically manufactured, and any of the essential fatty-acids such as Vitamin A, E, K.

As used herein, the term “volatile substance” refer to substances that readily evaporate into a gas under existing conditions. Examples may comprise those substances used in aromatherapy applications normally comprising essential volatile oils provided from, for instance, flowers, herbs and other selected plant life, fruits, etc. Oils derived from seeds such as grape seed, walnut, tea tree, or coffee bean cacao, or any compound in solid or powdered form that can be dissolved in a liquid—preferably a volatile oil—for rapid onset of action is also a potential use is inculcated.

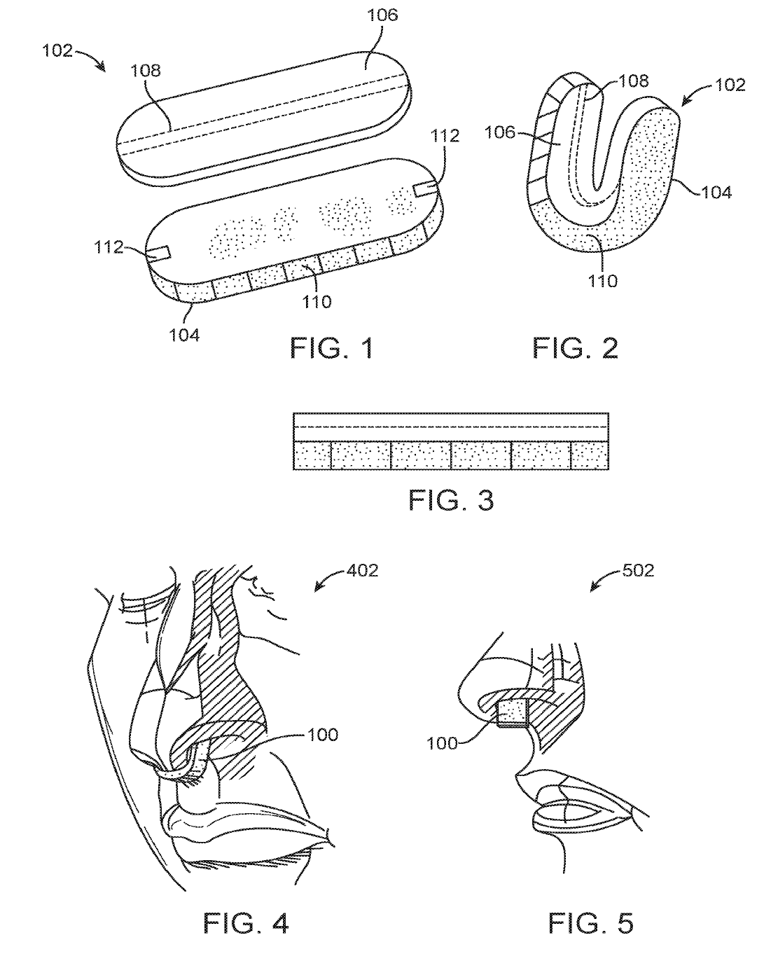

In exemplary embodiments, of the present invention, to be described in greater detail in relation to FIGS. 1-11.

The present invention provides, among other things, improved vehicles for and methods of introducing a vapor of one or more volatile and/or non-volatile substances into an inhalation flow path of a respiratory organ. Ensuing embodiments of the invention prove exemplary for aromatherapy application that are highly efficient, unobtrusive, easy to implement and require no specialized equipment or apparatus.

Turning now to the drawings in which like reference characters indicate corresponding elements throughout the several views, attention is first directed to FIG. 1 illustrating an exploded perspective view of an embodiment of a vehicle 102 for introducing a vapor of one or more volatile and/or non-volatile substances into an inhalation flow path of a user 402 (see FIG. 4) as the user 402 breathes.

The vehicle 102 comprises a series of layers including a carrier generally designated at 104, a barrier 106, and a clamp 108. Carrier 104 comprises, in this exemplary embodiment, a substantially elongated member 110 constructed of a natural or synthetic sponge-like or substantially absorbent material in the case where oils and liquids are used. The substantially elongate member may be freely removable by a user in case a solid substance such as a powder is used, as shown in FIG. 1. Protrusions 112 act. As such, in either circumstance, carrier 104 carries or is otherwise impregnated with one or more volatile and/or non-volatile substances. Barrier 106 has a clamp 108 in the form of a material or a pair of materials. The material may be disposed within the barrier 106 via a heat treatment. In this exemplary embodiment, no adhesives are used on the portion which contacts the user's skin such that only the clamp 108 is used as a means to secure the vehicle to the user's nasal cavity. Other means for clamp maybe provided such as metallic foil, wax paper, thin plastic or other material substantially impermeable to the one or more volatile and/or non-volatile substances carried by carrier 104. In this specific example, materials are used and comprise deformable or malleable metal or polyethylene.

In operation, when a volatile substance is employed, vehicle 102 may be grasped, such as by a human hand, positioned and deformed against the user's septum to clamp vehicle 102 against septum of nose in inhalation flow path as shown substantially in FIGS. 4 and 5. So installed, the volatility of the one or more volatile and/or non-volatile substances carried by carrier 104 will naturally emit a vapor which will introduce into inhalation flow path 25 to be carried into the lungs or respiratory tract as user 402.

On the other hand, in operation, when a non-volatile substance are employed, such as certain powders or gel-based microspheres, an activation step may be employed. Activation steps may comprise use of absorption promoter.

In this exemplary embodiment, a gelatin microcapsule, rather than being derived from animal or vegetable fat, is chemically produced and configured to hold the non-volatile substance (e.g., vitamin D). In operation, these microspheres can be infused the non-volatile substance, and also Nano-capsules such that a controlled release of both volatile, then non-volatile substance is achieved.

Referring now to FIG. 2, illustrated is a perspective view of the vehicle 102 for introducing a vapor of one or more volatile and/or non-volatile substances into an inhalation flow path as shown in FIG. 1, in its operational shape, in accordance with the present invention. FIG. 3 is a side view of the vehicle shown in FIGS. 1 and 2.

FIG. 4 is a perspective view of an embodiment of a vehicle for introducing a vapor of one or more volatile and/or non-volatile substances into an inhalation flow path, the vehicle shown as it would appear worn by a user, in accordance with the present invention, as shown at reference numeral 400.

FIG. 5 a perspective view of another embodiment of a vehicle for introducing a vapor of one or more volatile and/or non-volatile substances into an inhalation flow path, the vehicle shown as it would appear worn by a user, in accordance with the present invention, as shown at reference numeral 500.

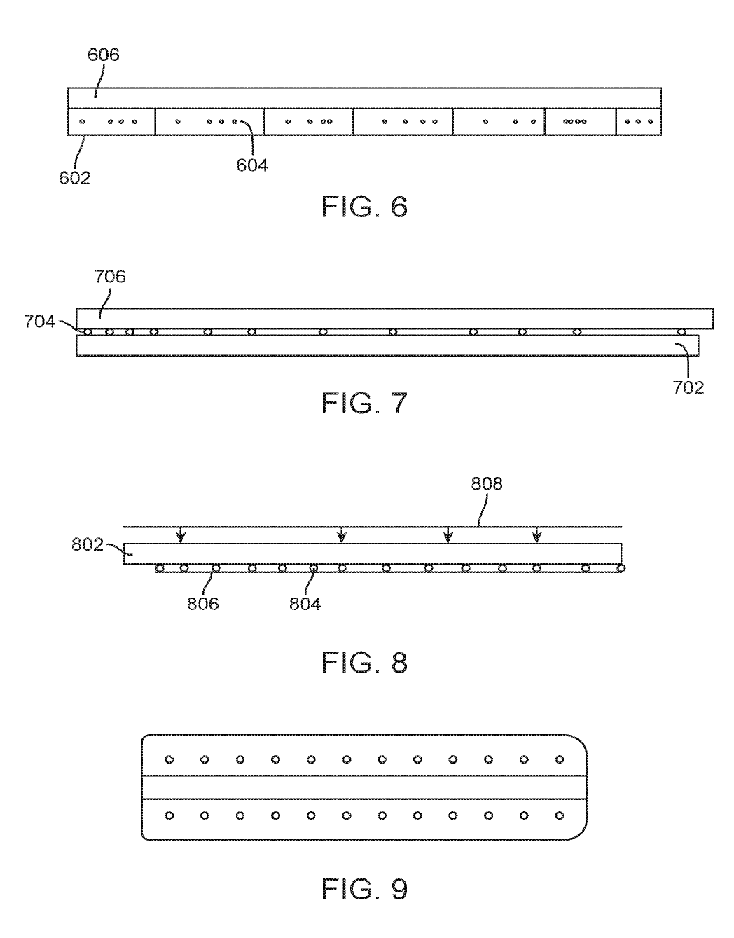

Now with reference to FIG. 6, a side view of a vehicle in accordance with an embodiment of the present invention is shown. As can be seen in this embodiment, prefilled carrier 602 includes microcapsules disposed within a conduit 604. In this way, the carrier is prefilled with materials during production. As such, a cover 606 is provided. The cover 606 is configured to seal the volatile or non-volatile material in so that the user can activate the vehicle released a material upon use. The cover 606 maybe comprise a polyester and may be heat sealed such that that it is user removable. In this way, a user can activate by peeling the cover back, assuming the substance is volatile and nature. If the substance is nonvolatile in nature, another activation step may be required. In optional embodiments, the user may fill the conduits with a chosen substance rather than being preloaded.

Referring now to FIG. 7, the carrier 702 maybe a solid form carrier having the volatile or nonvolatile substance coat to the surface of the carrier 702. In this exemplary embodiment microcapsules 704 coat the surface for our dispose on the surface such that they are operationally attached thereto. The cover seven 706 similar in nature to cover 606 may be provided to cover the microbeads on the surface of the carrier 702. Like the cover of FIG. 6, the cover maybe comprised of polyester maybe heat shield and maybe simply peeled back prior to use by the user.

Referring now to FIG. 8, in an optional embodiments of the present invention, the vehicle 110 maybe translucent for nearly invisible look. In this embodiment, a clear colorless polyester sheet is provided at reference number 802. On one side of the polyester sheet, microcapsules with a binder 804 are dispose on or impregnated within the polyester sheet. A perimeter heat seal 806 it is further provided and in disposed on the surface of the polyester sheet to shield the microcapsules. Polyethylene coated materials 808 may be disposed on the other side of the polyester sheet and provide a clamp for the nasal passages of a user.

Presenting the volatile and/or non-volatile substances in the form of microcapsules make it possible to conserve the volatile and/or non-volatile substance under optimum conditions prior to use, to use a wide range of volatile and/or non-volatile substances almost independently of any chemical compatibility with the carrier, given that the volatile and/or non-volatile substance is isolated within the walls of the microcapsules prior to being diffused in the air; and to use methods that are relatively easy to implement and of low cost when depositing microcapsules on the carrier.

The volatile and/or non-volatile substance contained in the microcapsules are selected from substances that are liposoluble and substances that are hydrosoluble. In optional embodiments, the microcapsules have a thin outer wall made of a material selected from porous aminoplast polymers, polyamide polymers, polyurethane polymers, and cellulose polymers; the microcapsules adhere to the filter material by chemical compatibility; the microcapsules adhere to the filter material by means of a chemical binder; and the binder is selected from polyurethane binders, polyamide binders, silicone binders, acrylic binders, and epoxy binders.

The invention also provides a method of manufacturing a vehicle as defined above, in which method the microcapsules are deposited on the carrier medium by means of a method selected from: padding; coating; spraying; bath depletion; silk-screen printing; passing the carrier material over at least one cylinder turning in a liquid containing microcapsules; and offset printing.

In conventional manner, the microcapsules are made by forming a polymerized wall around droplets or particles of the volatile or non-volatile substance to be diffused in the air. The wall material of the microcapsules is adapted to release the encapsulated volatile substance progressively by diffusion through the porous wall of the microcapsule (where appropriate under the effect of a small amount of pressure, several millibars, due to air passing through the filter), or where appropriate by breaking said wall.

Advantageously, the polymer forming the thin wall of each microcapsule can be made of porous aminoplast, of polyamide, or polyurethane, or where appropriate of a cellulose polymer such as ethyl cellulose or cellulose acetate butyrate.

Advantageously, the microcapsules can be grafted onto the fibers of the carrier material by chemical bonds, in particular by means of a polyurethane, polyamide, silicone, acrylic, or epoxy binder.

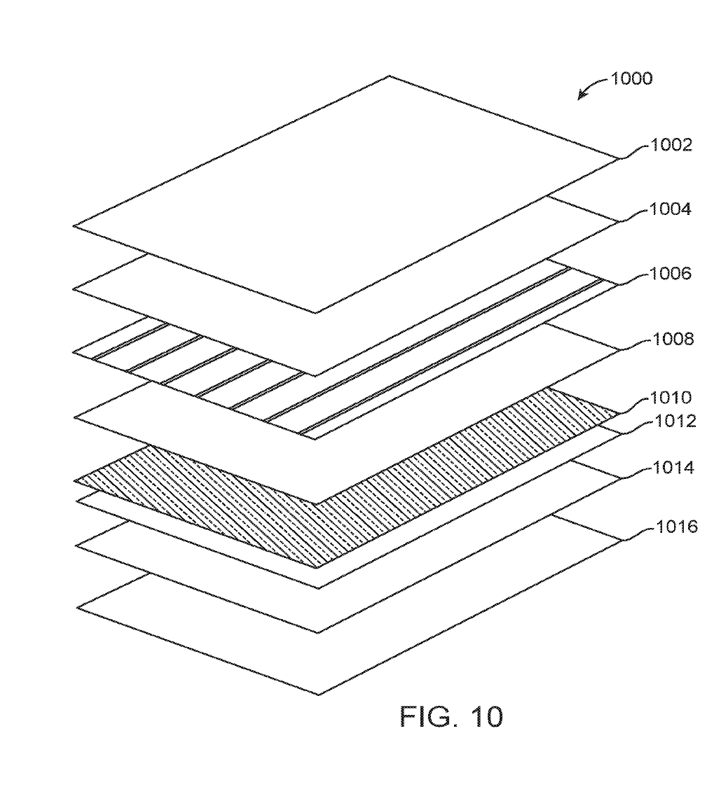

Now with reference to FIG. 10, an exploded perspective view of an embodiment of the present invention is shown generally at 1000. The vehicle 1000 comprises a top synthetic paper layer 1000, underneath which is a layer of PSA 1004, underneath which is a layer of flexible composite material 1006. A second layer of PSA is provided at 1008. A layer of top substrate 1010 and a bottom layer of substrate 1012 is provided above a slurry layer 1014. A bottom layer 1016 is further provided.

Synthetic paper layer 1002 may comprise Polyart or environmentally friendly synthetic paper is made by Arjobex. A flexible composite material 1006 is sandwiched between two layers of PSA 1004 and 1008 and configured as a clamp.

Now with reference to FIG. 10, an optional embodiment administering substances into an innovation flow position generally at 1000.

A top and bottom layers of substrate 1010, 1012, may comprise, for example, Teslin® substrate, and be configured with microporosity, in some embodiments. A slurry layer 1014 is provided under a more durable bottom layer 1016.

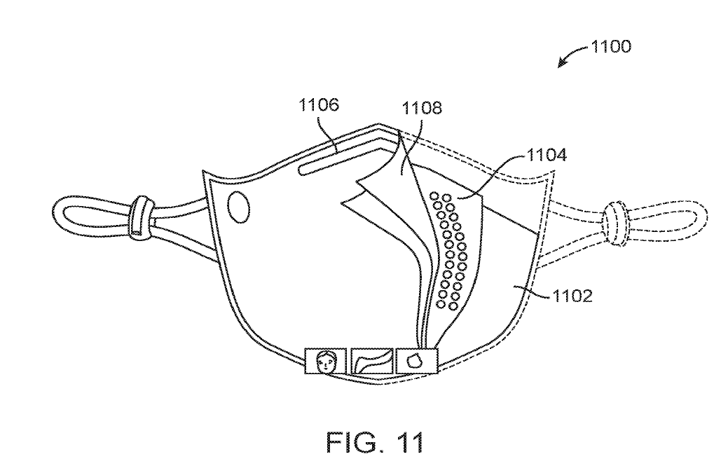

Now with reference to FIG. 11, an optional embodiment administering substances into an innovation flow position generally at 1100. As shown the vehicle 1100 is a surgical style mask. The Vehicle 1100 comprises in inner layer 1102, a middle material 1104, and an outer layer 1106.

The base layer 1102 accesses the barrier between the vehicle and the users skin. The middle layer is configured for access to the carrier, and as such, can be impregnated with microcapsules, powder, or liquid substances that are volatile and nonvolatile alike, and mixtures thereof, may be disposed atop or within the middle layer carrier 1104. Application of pressure for the production of heat may activate substance for consumption. Each of the layers shown in FIG. 10 may be disposed in the vehicle of FIG. 11 as well.

In summary, the present invention provides exemplary vehicles and methodologies for introducing a vapor of one or more volatile and/or non-volatile substances into an inhalation flow path of a respiratory organ in aromatherapy application. Foregoing embodiments of the present invention are easy to construct, and provide a variety of means for providing or otherwise impregnating a selected vehicle with one or more volatile and/or non-volatile substances. In this regard, the various embodiments are not only convenient, but also permit users to easily transport aromatherapy vehicles for selective and desired use as needed without having to employ specialized equipment.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim. While the present invention has been described in connection with what are presently considered to be the most practical and preferred embodiments, it is to be understood that the present invention is not limited to these herein disclosed embodiments. Rather, the present invention is intended to cover all of the various modifications and equivalent arrangements included within the spirit and scope of the appended claims.

Although specific features of various embodiments of the invention may be shown in some drawings and not in others, this is for convenience only. In accordance with the principles of the invention, the feature(s) of one drawing may be combined with any or all of the features in any of the other drawings. The words “including”, “comprising”, “having”, and “with” as used herein are to be interpreted broadly and comprehensively and are not limited to any physical interconnection. Moreover, any embodiments disclosed herein are not to be interpreted as the only possible embodiments. Rather, modifications and other embodiments are intended to be included within the scope of the appended claims.

1. A method of introducing a vapor of at least one substance into an inhalation flow path of a respiratory organ, the method comprising the steps of:
providing a carrier proximate to a surface of skin;
providing the carrier with at least one substance;
providing a barrier coupled to a surface of the carrier, the barrier being substantially impermeable to the substances carried by the carrier;
providing a clamp disposed within or atop the barrier and configured as a clamp for a septum of a user, wherein the clamp comprises a polyethylene coated material heat sealed within the barrier, wherein the polyethylene coated material is further coated with the substance; and
engaging the carrier to the surface of skin proximate an inhalation flow path of a respiratory organ with the barrier interposed between the carrier and the skin to prevent the one or more substances from contacting the skin;
wherein the substance is non-volatile.

2. The method of claim 1, wherein the step of providing a carrier further comprises the step of providing a substantially absorbent member, wherein the substantially absorbent member is held therein by at least one protrusion located on inward facing wall of the carrier.

3. The method of claim 1, wherein the step of providing the carrier with at least one substance further includes the step of impregnating the carrier with microcapsules.

4. The method of claim 3, wherein the step of impregnating the carrier with at least one substance further comprises the steps of:
providing the microcapsule with a mixture of a volatile and the non-volatile substance; and
rupturing the microcapsule capsule using heat or pressure.

5. A vehicle for introducing a vapor of one at least substance into an inhalation flow path of a respiratory organ, the vehicle comprising:
a carrier conformable to a surface of skin;
at least one substance carried by the carrier;
a barrier coupled to a surface of the carrier, the barrier being substantially impermeable to the at least substance carried by the carrier; and
a clamp disposed within or atop the barrier and configured as a clamp for a septum of a user, wherein the clamp comprises a polyethylene coated material is heat sealed within the barrier;
wherein the polyethylene coated material is further coated with the substance;
wherein the substance is non-volatile.

6. The vehicle of claim 5, wherein the carrier comprises a substantially absorbent member, wherein the substantially absorbent member is held therein by at least one protrusion located on inward facing wall of the carrier.

7. The vehicle of claim 5, wherein the carrier is impregnated with with microcapsules during production.

8. The vehicle of claim 7, wherein the microcapsule are a mixture of a volatile and the non-volatile substance and are rupturable with the application of heat or pressure.

9. (canceled)

10. (canceled)

11. (canceled)

12. (canceled)

13. (canceled)

14. (canceled)

15. (canceled)
